(19) United States
(12) Patent Application Publication
(10) Pub. No.: US 2010/0185083 A1
(43) Pub. Date: Jul. 22, 2010

(54) NAVIGATION ENABLED LEAD DELIVERY CATHETER

(75) Inventors: Michael R. Neidert, Co. Galway (IE); Niall Duffy, Tuam (IE)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/643,737

(22) Filed: Dec. 21, 2009

Related U.S. Application Data
(63) Continuation-in-part of application No. 12/275,405, filed on Nov. 21, 2008.

(51) Int. Cl. A61B 5/05; A61B 19/00

(52) U.S. Cl. 600/424; 606/129

(57) ABSTRACT

A catheter system for positioning of a medical instrument includes a first elongate member having a first lumen. The system also includes a second elongate member that includes a second lumen that receives the medical instrument. The second elongate member is coupled directly to the first elongate member, and the second elongate member is cuttable to allow removal of the medical instrument from the second lumen. The second elongate member is also selectively collapsible and expandable to change a size of the second lumen. Furthermore, the system includes a navigation tool operable for allowing detection of a location of the catheter system. The navigation tool is received in the first lumen, and the second elongate member is cuttable without cutting the first elongate member and the navigation tool.

### CROSS-REFERENCE TO RELATED APPLICATIONS

This application is a continuation-in-part of U.S. patent application Ser. No. 12/275,405 filed on Nov. 21, 2008, the entire disclosure of which is incorporated herein by reference.

### FIELD

The present disclosure relates generally to catheter systems and, more specifically, relates to a navigation enabled lead delivery catheter.

### BACKGROUND

The statements in this section merely provide background information related to the present disclosure and may not constitute prior art.

Various devices have been proposed for introducing and/or positioning elongate medical instruments in the human body. For instance, various catheter systems, introducer sheaths, and other elongate tubular members have been proposed for these purposes. More specifically, these devices can include a proximal portion that remains outside the body and a tubular member that extends into the body such that a distal end of the tubular member is located at a desired position. A medical instrument, such as a cardiac pacemaker lead or other vascular instrument, passes longitudinally through a lumen of the tubular member toward the desired position. For instance, in some embodiments, the medical instrument extends through the lumen to be positioned within the patient's heart, blood vessel, or other portion of the body. Then, the medical professional removes the tubular member of the catheter system, leaving the medical instrument in its intended position within the body. For example, the catheter system can be cut or peeled longitudinally as it is pulled from the body, thereby allowing the medical instrument to move outside the lumen of the catheter system, and leaving the distal end of the medical instrument in its intended position within the body.

Although conventional catheter systems have functioned for the intended purposes, some problems remain. For instance, it can be difficult to properly locate and position the distal end of the catheter system into the desired position within the body. As such, the medical instrument may be extended through the lumen of the catheter system into an incorrect position within the body, and the medical instrument may malfunction and/or need to be repositioned.

For instance, medical professionals typically manipulate the proximal end of the catheter system by pushing, pulling, and twisting the proximal end, and these forces are transferred longitudinally along the catheter system to thread the distal end into the desired position within the body. However, some catheter systems may not provide sufficient structural rigidity to properly transfer the forces from the proximal end to the distal end, thereby hindering the intended movement of the distal end.

Moreover, because the distal end is within the body, and the medical professional cannot view the distal end directly, it can be difficult to position the distal end with a high degree of accuracy. In some cases, navigation tools are used to more accurately locate the distal end. More specifically, these tools can include a coil of wire that is wound around the outside of the distal end. Then, a current is induced within the coil by generating an electromagnetic field from outside the patient. This induced current is detected in order to triangulate and locate the distal end within the body. However, in cases where the catheter system is split longitudinally after the medical instrument is placed, the coil may need to be segmented and the patient's exposure to the material of the coil should be limited, which can be problematic.

### SUMMARY

This section provides a general summary of the disclosure, and is not a comprehensive disclosure of its full scope or all of its features.

A catheter system for positioning of a medical instrument is disclosed that includes a first elongate member having a first lumen. The system also includes a second elongate member that includes a second lumen that receives the medical instrument. The second elongate member is coupled directly to the first elongate member, and the second elongate member is cuttable to allow removal of the medical instrument from the second lumen. The second elongate member is also selectively collapsible and expandable to change a size of the second lumen. Furthermore, the system includes a navigation tool operable for allowing detection of a location of the catheter system. The navigation tool is received in the first lumen, and the second elongate member is cuttable without cutting the first elongate member and the navigation tool.

A method of manufacturing a catheter system is also disclosed. The method includes directly coupling an outer surface of a first elongate member having a first lumen to an outer surface of a second elongate member having a second lumen such that a longitudinal axis of the first elongate member extends substantially in the same direction as a longitudinal axis of the second elongate member. The method further includes reducing a size of the second elongate member from an expanded state to a collapsed state to reduce a size of the second lumen. Moreover, the method includes retaining the second elongate member in the collapsed state.

Still further, a catheter system for positioning of a medical instrument is disclosed. The catheter system includes a first elongate member that includes a first lumen and an outer surface. The system also includes a reinforcing member that reinforces the first elongate member. Moreover, the system includes a navigation tool with an electrically conductive coil operable for detecting a location of the catheter system. The navigation tool is received in the first lumen, and the navigation tool includes an electrically conductive coil in which a current is induced in the presence of an electromagnetic field. The current is dependent on the location of the catheter system. Furthermore, the system includes a second elongate member that includes a second lumen that receives the medical instrument and an outer surface. The outer surface of the second elongate member is longitudinally coupled directly to the outer surface of the first elongate member. Also, the second elongate member is cuttable generally along a second longitudinal axis thereof to allow removal of the medical instrument from the second lumen. The second elongate member is cuttable without cutting the first elongate member, the reinforcing member, and the navigation tool. In addition, the second elongate member is selectively collapsible and expandable to change a size of the second lumen. Still further, the system includes a port member having a first port in fluid communication with the first lumen and a second port in fluid communication with the second lumen. The navigation tool extends through the first port and into the first lumen, and the medical instrument is extendable through the second port and into the second lumen.

Further areas of applicability will become apparent from the description provided herein. The description and specific examples in this summary are intended for purposes of illustration only and are not intended to limit the scope of the present disclosure.

### DRAWINGS

The drawings described herein are for illustration purposes only and are not intended to limit the scope of the present disclosure in any way.

FIG. 1A is a perspective view of a catheter system according to various teachings of the present disclosure illustrated with a first elongate member and a second elongate member that is in a collapsed state;

FIG. 1B is a perspective view of the catheter system of FIG. 1A with the second elongate member illustrated in an inflated state;

FIG. 2 is a perspective view of a port member of the catheter system of FIGS. 1A and 1B;

FIG. 3 is a perspective view of the catheter system of FIG. 1B with a navigation tool and a medical instrument extending therethrough;

FIG. 4 is a perspective view of the catheter system of FIG. 1B shown during a cutting operation;

FIG. 5 is a sectional view of the catheter system of FIG. 1A shown during a manufacturing operation thereof;

FIG. 6 is a sectional view of the catheter system taken along the line 6-6 of FIG. 1B; and

FIG. 7 is a sectional view of the catheter system taken along the line 7-7 of FIG. 1B.

### DETAILED DESCRIPTION

The following description is merely exemplary in nature and is not intended to limit the present disclosure, application, or uses. It should be understood that throughout the drawings, corresponding reference numerals indicate like or corresponding parts and features.

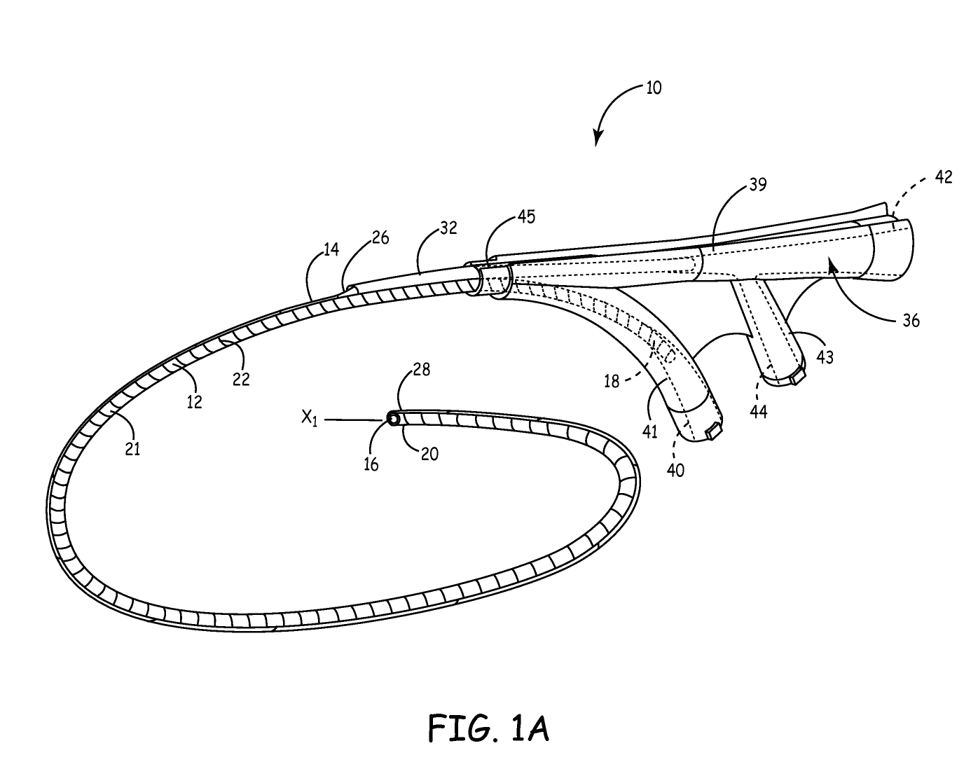

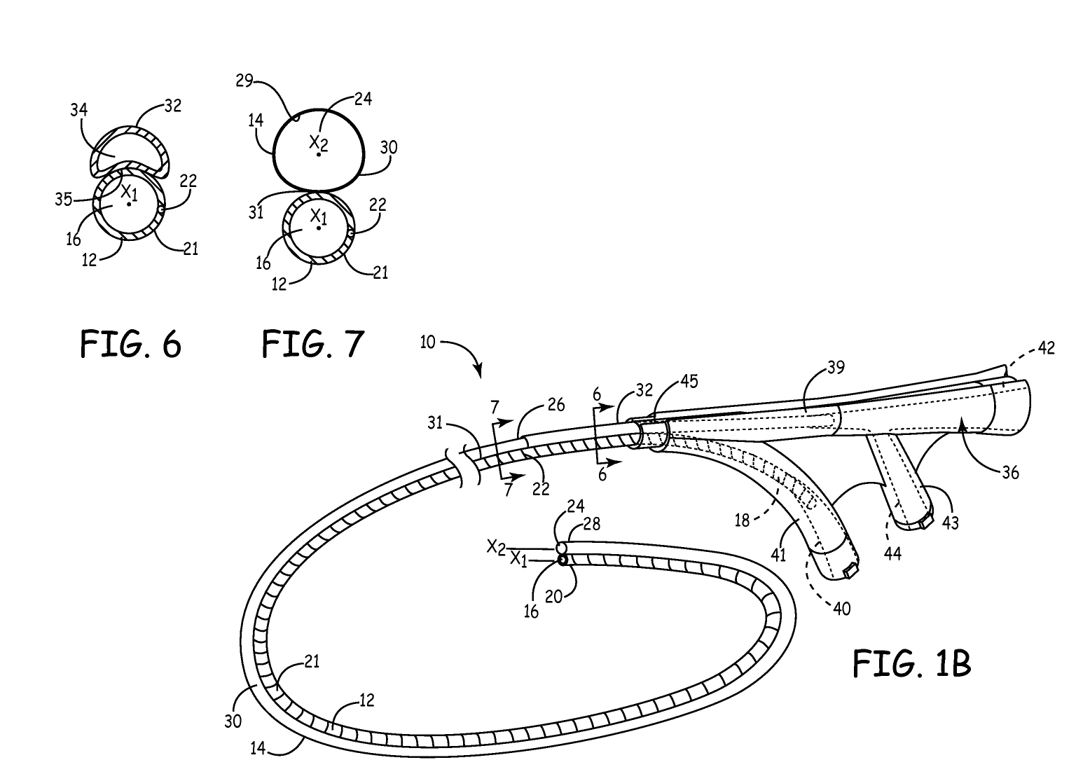

Referring initially to FIGS. 1A, 1B, 6 and 7, a catheter system 10 according to various exemplary embodiments is illustrated. The catheter system 10 can include a first elongate member 12 and a second elongate member 14. In one or more embodiments, first elongate member 12 provides structural rigidity, torque, pushability, whereas second elongate member 14 provides a channel for the lead as well as an easy path for slitting. The second elongate member 14 can be selectively collapsible and expandable. More specifically, FIG. 1A illustrates one exemplary embodiment of the second elongate member 14 in a collapsed state, and FIG. 1B illustrates one exemplary embodiment of the second elongate member 14 in an expanded state. As will be discussed in greater detail below, the collapsibility and expandability of the second elongate member 14 can facilitate use of the catheter system 10 for added convenience.

The first elongate member 12 can be tubular, hollow, and generally flexible. As such, the first elongate member 12 can include a first lumen 16 (FIG. 6). Also, the first elongate member 12 can include a first longitudinal axis X₁. Moreover, the first elongate member 12 can include a proximal end 18, a distal end 20, and an outer surface 21.

The first elongate member 12 can be made out of any suitable material and can have any suitable construction. For instance, in some exemplary embodiments, the first elongate member 12 can be made from a metallic braided shaft or a spiral cut hypotube, such as a DRIVER or MX hypotube, which is commercially available from Medtronic, Inc. of Minneapolis, Minn. Alternatively, a braided shaft is commercially available from Accellent located in Minneapolis, Minn. or TFX OEM located in Düsseldorf, Germany while a spiral cut hypotube is commercially available from Creganna located in Minneapolis, Minn. As such, the first elongate member 12 can be made out of metal (e.g., Nitinol or stainless steel). Also, the first elongate member 12 can be coated or otherwise surrounded by a relatively thin polymeric material in order to limit exposure of the patient to the metallic material of the hypotube. Exemplary polymeric material that can be used to form first elongate member 12 are polyimide, polyether ether ketone (PEEK) and/or polyether block amide (PEBAX).

Moreover, in some exemplary embodiments, the first elongate member 12 can be made from a polymeric tube, such as a tube made of PEBAX with a durometer between about 35 D and 62 D. As such, the first elongate member 12 can additionally include a reinforcing member 22 (FIGS. 1A and 6). The reinforcing member 22 can generally reinforce the first elongate member 12. Accordingly, in some embodiments, the reinforcing member 22 can be embedded in the first elongate member 12. More specifically, in some exemplary embodiments, the reinforcing member 22 can be helical and can wind about the first axis X₁ in a helical manner. In other embodiments, the reinforcing member 22 can be a braided member such that the reinforcing member 22 includes a plurality of helical members that helically wind about the first axis X₁ and that intersect each other along the axis X₁. The reinforcing member 22 can be made of any suitable material, such as metal (e.g., nitinol or stainless steel).

It will be appreciated that the reinforcing member 22 reinforces the first elongate member 12 to increase the rigidity of the first elongate member 12 such that forces applied at the proximal end 18 (e.g., forces exerted along the axis X₁ and/or rotational forces exerted about the axis X₁) can be transferred to the distal end 20 due to the reinforcement supplied by the reinforcing member 22. Accordingly, the first elongate member 12 can be more easily manipulated, routed along a non-linear path (e.g., a blood vessel), and the like.

Furthermore, in some embodiments, the second elongate member 14 can be tubular and hollow so as to define a second lumen 24 (FIG. 7) extending along a second longitudinal axis X₂. Furthermore, the second elongate member 14 can include a proximal end 26 and a distal end 28. Additionally, the second elongate member 14 can include an inner surface 29 and an outer surface 30.

The second elongate member 14 can be made of any suitable material, such as a polymeric material. Exemplary polymeric material that can be used for the second elongate member 14 can include polyurethane, PEBAX and/or polyethylene.

In some embodiments, the second elongate member 14 can be made of a material similar to that of a known percutaneous transluminal coronary angioplasty (PTCA) balloon. In one or more embodiments, it is preferable to form the second elongate member 14 of a different material than the first elongate member 12. In one or more other embodiments, the second elongate member 14 is formed of the same material as the first elongate member 12.

As is best illustrated in FIGS. 1B and 7, the second elongate member 14 can be longitudinally coupled directly to the first elongate member 12. More specifically, the outer surface 30 of the second elongate member 14 can be fixed to the outer surface 21 of the first elongate member 12 such that the second axis X₂ extends substantially in the same direction as the first longitudinal axis X₁. It will be appreciated that the first and second elongate members 12, 14 could be coupled in any suitable manner. For instance, in some exemplary embodiments, the first and second elongate members 12, 14 can be fixed together with an adhesive 31 that extends along the axis X₁, X₂. In other exemplary embodiments, the first and second elongate members 12, 14 can be over-molded together.

The second elongate member 14 can be configured to be selectively collapsible and expandable to change the size of the second lumen 24 within the second elongate member 14. More specifically, in some exemplary embodiments, the second elongate member 14 can have a thin wall thickness as compared with the first elongate member 12. For instance, in some exemplary embodiments, the second elongate member 14 can have a wall thickness between about 0.0007″ and 0.0015″ while the wall thickness of the first elongate member 12 can be about 0.01″.

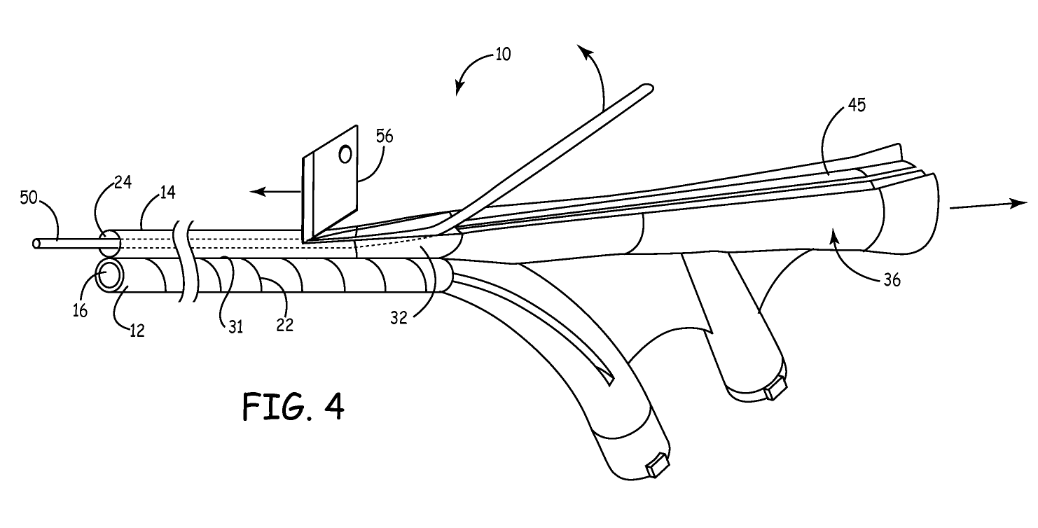

Furthermore, as will be described in greater detail below in relation to FIG. 4, the second elongate member 14 can be configured to be at least partially segmented generally along the second longitudinal axis X₂. For instance, as will be described in greater detail below, the second elongate member 14 can be cut so as to provide access into and out of the second lumen 24. Also, as will be discussed below, because the second elongate member 14 is collapsible and expandable and segmentable, the catheter system 10 can be more convenient during use.

Moreover, in some exemplary embodiments, the catheter system 10 can include an end member 32 (FIGS. 1A, 1B, 6). The end member 32 can be tubular and hollow so as to define a third lumen 34 (FIG. 6). The end member 32 can be coupled to the proximal end 26 of the second elongate member 14 such that the third lumen 34 is in fluid communication with the second lumen 24 of the second elongate member 14. Also, the end member 32 can be longitudinally coupled and fixed to the outer surface 21 of the first elongate member 12. In some exemplary embodiments shown in FIG. 6, the end member 32 can include a recess 35 along the entire longitudinal length of the end member 32, and the recess 35 receives a portion of the first elongate member 12. It will be appreciated that the end member 32 can be coupled to the first elongate member 12 and the second elongate member 14 in any suitable fashion. For instance, in some exemplary embodiments, the end member 32 can be fixed to the elongate members 12, 14 via an adhesive. In other exemplary embodiments, the end member 32 can be over-molded to the first elongate member 12 and/or the second elongate member 14.

The end member 32 can be made of a material more resistant to deformation (i.e., more rigid) than that of the second elongate member 14. More specifically, the end member 32 can be configured such that the third lumen 34 retains substantially the same shape regardless of whether the second elongate member 14 is collapsed or expanded. Also, in some exemplary embodiments, the end member 32 can be made of a polymeric material and can have a wall thickness of about 0.004″.

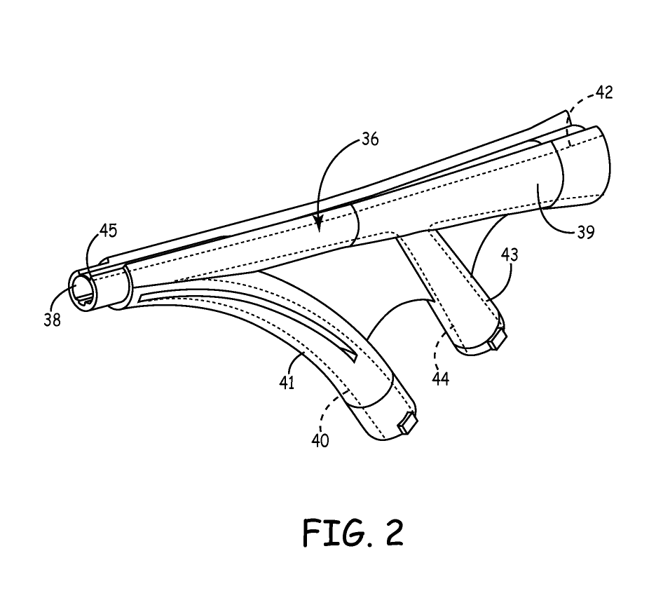

Furthermore, in the exemplary embodiments shown in FIGS. 1A, 1B, and 2), the catheter system 10 can include a port member 36. Generally, the port member 36 can include a main body 39, a first branch 41, and a second branch 43 disposed between the main body 39 and the first branch 41. Also, in some exemplary embodiments, the port member 36 can include a first port 40, which extends through the first branch 41 and is in fluid communication with a common aperture 38. Additionally, the port member 36 can include a second port 42, which extends through the main body 39 and is in fluid communication with the common aperture 38. Moreover, the end member 36 can include a third port 44, which extends through the second branch 43 and is in fluid communication with the second port 42.

The port member 36 can be made out of any suitable material, such as a polymeric material. Also, the port member 36 can be generally rigid. Additionally, the port member 36 can include a thin walled portion 35 which extends longitudinally along the main body 39. As will be discussed in greater detail below, the thin walled portion 45 can be segmented (e.g., cut). The port member 36 can also include one or more coupling members, such as luer locks or other couplings disposed adjacent respective ends of the first branch 41, the second branch 43, and the main body 39.

The port member 36 can also be operatively coupled to the first and second elongate members 12, 14 as well as the end member 32. For instance, in some exemplary embodiments, the end member 32 is received within the common aperture 38, and the end member 32 extends into the main body 39 (FIGS. 1A and 1B). In some exemplary embodiments, the second and third ports 42, 44 are in fluid communication with the third lumen 34 of the end member 32 and, thus, the second lumen 24 of the second elongate member 14. Moreover, the proximal end 18 of the first elongate member 12 can be received within the common aperture 38 and into the first port 40 such that the first port 40 is in fluid communication with the first lumen 16 of the first elongate member 12.

It will be appreciated that the port member 36 can be coupled to the end member 32, the first elongate member 12, and the second elongate member 14 in any suitable fashion. For instances, in some exemplary embodiments, an adhesive is used for these purposes. In other exemplary embodiments, the port member 36 is over-molded to the end member 32 and/or the first elongate member 12.

It will also be appreciated that the end member 32 is optionally included in the catheter system 10. For instance, in some exemplary embodiments, the second elongate member 14 can be coupled directly to the port member 36 such that the second elongate member 14 is received within the common aperture 38 and such that the second and third ports 42, 44 are in fluid communication with the second lumen 24 of the second elongate member 14. Moreover, it will be appreciated that the third port 44 and the second branch 43 are optional. For instance, in some exemplary embodiments, the port member 36 includes only the main body 39 and the first branch 41.

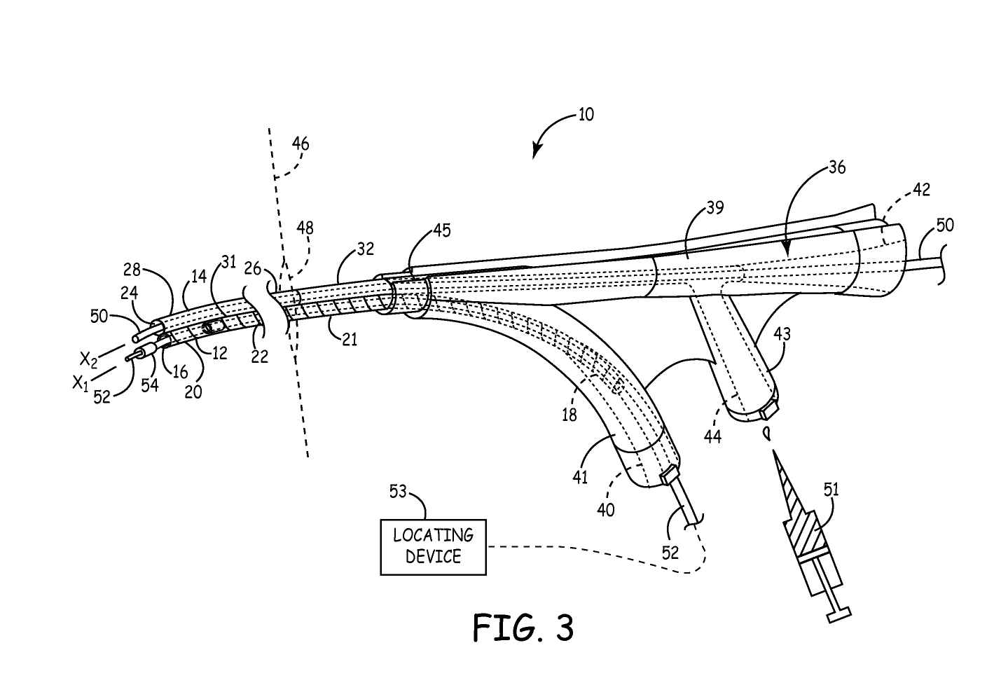

Accordingly, operation of the catheter system 10 will be described in greater detail. As shown in FIG. 3, the first and second elongate members 12, 14 are inserted into a patient (shown schematically at 46) through a prepared incision 48. In some exemplary embodiments, the second elongate member 14 is collapsed as shown in FIG. 1A as the catheter system 10 is inserted through the incision 48. Since the second elongate member 14 is collapsed, the catheter system 10 can have a lower profile and can be more flexible to facilitate moving the first and second elongate members 12, 14 to a target location within the patient 46. Also, in some exemplary embodiments, the outer surface 21, 30 of the first and second elongate members 12, 14 are at least partially coated with a lubricant to reduce friction as the first and second elongate members 12, 14 are inserted into the incision 48 and threaded into the patient 46.

Furthermore, in some exemplary embodiments, the location of the distal end 20 of the first elongate member 12 is detected to ensure that the distal end 20 is in a predetermined position within the patient 46. In some embodiments, placement and location of the distal end 20 within the patient 46 is performed using a navigation tool 52. It will be appreciated that the navigation tool 52 could be used to detect the position of any suitable portion of the catheter system 10.

In some exemplary embodiments, the navigation tool 52 can be elongate and can include an electrically conductive coil 54 on one end thereof. The navigation tool 52 can be of any suitable type, such as the navigation tool 52 disclosed in Applicant's co-pending U.S. patent application Ser. No. 12/275,377 filed Nov. 21, 2008, which is incorporated herein by reference in its entirety. Thus, the navigation tool 52 can be used in conjunction with a locating device 53. The locating device 53 can include microprocessors, computer memory, and other computer components for calculating and detecting the position of the coil 54, and thus, the position of the navigation tool 52 within the patient 46. Also, the locating device 53 can be used in combination with Johnson & Johnson's CARTO XP EP Navigation and Ablation System, commercially available from Johnson & Johnson of New Brunswick, N.J. Furthermore, the locating device 53 can be used in combination with the FLUOROMERGE or AXIEM Electromagnetic Tracking Systems, commercially available from Medtronic Surgical Navigation Technologies, Inc. of Minneapolis, Minn.

Accordingly, the locating device 53 can generate a variable electromagnetic field about the patient 46, and the electromagnetic field can induce a current within the coil 54 of the navigation tool 52. The induced current is dependent upon the position of the coil 54 relative to the locating device and within the electromagnetic field. Accordingly, the locating device 53 can triangulate the coil 54 to, thereby, detect the position of the navigation tool 52 within the patient 46. Also, the locating device 53 can rely on imaging technology (X-ray, MRI, CT, etc.) to produce visual feedback of the position of the navigation tool 52 within the patient 46.

Thus, the navigation tool 52 can be inserted into the first elongate member 12 of the catheter system 10 via the second port 42 of the port member 36. More specifically, the navigation tool 52 can be extended into the first port 40, into the first lumen 16, and threaded along the first axis X₁. It will be appreciated that the navigation tool 52 could be inserted into the first elongate member 12 either before or after the first elongate member 12 is inserted into the patient 46. In either case, the navigation tool 52 can be used to detect the position of the distal end 20 of the first elongate member 12 and to move the distal end 20 into a predetermined position within the patient 46. It will be appreciate that the distal end 28 of the second elongate member 14 can be disposed in a predetermined and known position relative to the distal end 20 of the first elongate member 12, and as such, the navigation tool 52 can be used to similarly move the distal end 28 of the second elongate member 14 into a predetermined position within the patient 46.

Also, in some exemplary embodiments, a working fluid 51 is then introduced into the second lumen 24 via the third port 44 of the port member 36. The working fluid 51 flows into the second lumen 24 and applies fluid pressure against the inner surface 29 of the second lumen 24 to expand the second elongate member 14 from a collapsed state (FIG. 1A) to an expanded state (FIG. 1B). The working fluid 51 could be of any suitable type, such as saline.

Once the second elongate member 14 is sufficiently expanded, the medical instrument 50 can be inserted into the second lumen 24. More specifically, the medical instrument 50 is threaded into the second port 42 of the port member 36, through the third lumen 34 of the end member 32, and is received in the second lumen 24 of the second elongate member 14. The medical instrument 50 can be threaded along the second lumen 24 until it extends out of the distal end 28 of the second elongate member 14 toward a desired location within the patient. Then, in some exemplary embodiments, the medical instrument 50 can be attached to tissue (not shown) of the patient 46 in any suitable, known fashion.

It will be appreciated that the medical instrument 50 could be of any suitable type. For instance, in some exemplary embodiments, the medical instrument 50 can be a cardiac lead (e.g., pacemaker or defibrillator lead) that transmits an electrical signal to and/or from cardiac tissue (not shown) In other exemplary embodiments, the medical instrument 50 can be a neural lead that transmits an electrical signal to and/or from neural tissue (not shown).

In another embodiment, the working fluid 51 is not used to expand the second elongate member 14, and instead, insertion of the medical instrument 50 causes expansion of the second elongate member 14. More specifically, as the medical instrument 50 progressively extends along the axis X₂, the medical instrument 50 pushes the inner surface 29 of the second elongate member 14 outward radially to expand the second elongate member 14. Also, the inner surface 29 of the second lumen 24 can include a lubricant for reducing friction and facilitating movement of the medical instrument 50 along the axis X₂ of the second elongate member 14.

Once the medical instrument 50 is in the desired position with the patient 46, the catheter system 10 can be removed from the medical instrument 50. More specifically, in some exemplary embodiments illustrated in FIG. 4, a blade 56 can be used to at least partially segment (e.g., cut) the port member 36, the end member 32, and the second elongate member 14. In some embodiments, the port member 36 is pulled away from and out of the patient 46 to withdraw the first and second elongate members 12, 14, and the blade 56 simultaneously cuts (i.e. slits) longitudinally along the thin walled portion 45 of the port member 36, along the end member 32, and through the wall thickness of the second elongate member 14 generally parallel to the second longitudinal axis X₂. As such, the medical instrument 50 can be moved out of the port member 36, the end member 32, and the second lumen 24 of the second elongate member 14. Accordingly, the catheter system 10 is removed from the patient 46 while the blade 56 simultaneously cuts, and the catheter system 10 can be removed from the medical instrument 50, leaving the medical instrument 50 in its desired position within the patient 46.

It will be appreciated that the port member 36, the end member 32, and/or the elongate member 14 can be segmented in any suitable fashion. For instance, in other exemplary embodiments, the second elongate member 14 is scored generally along the second axis X₂, such that the second elongate member 14 can be peeled along the axis X₂ along the scoring without the use of the blade 56.

It will also be appreciated that the catheter system 10 can be segmented in any direction other than along the second axis X₂. Moreover, it will be appreciated that the catheter system 10 can be only partially segmented along the axis X₂ in order to remove the catheter system 10 from the medical instrument 50.

Accordingly, the catheter system 10 allows the medical instrument 50 to be accurately positioned within the patient 46 such that the medical instrument 50 is more likely to operate in an intended fashion. Also, because the medical instrument 50 is isolated from the navigation tool 52, the medical instrument 50 can be easily removed from the catheter system 10 simply by cutting through the relatively thin wall of the second elongate member 14. Furthermore, exposure to the material of the coil 54 of the navigation tool 52 is unlikely because the navigation tool 52 remains uncut (i.e., it is not segmented) during removal of the catheter system 10. Likewise, exposure to the material of the reinforcing member 22 of the first elongate member 12 is unlikely because the first elongate member 12 and reinforcing member 22 remain uncut (i.e., not segmented) during removal of the catheter system 10.

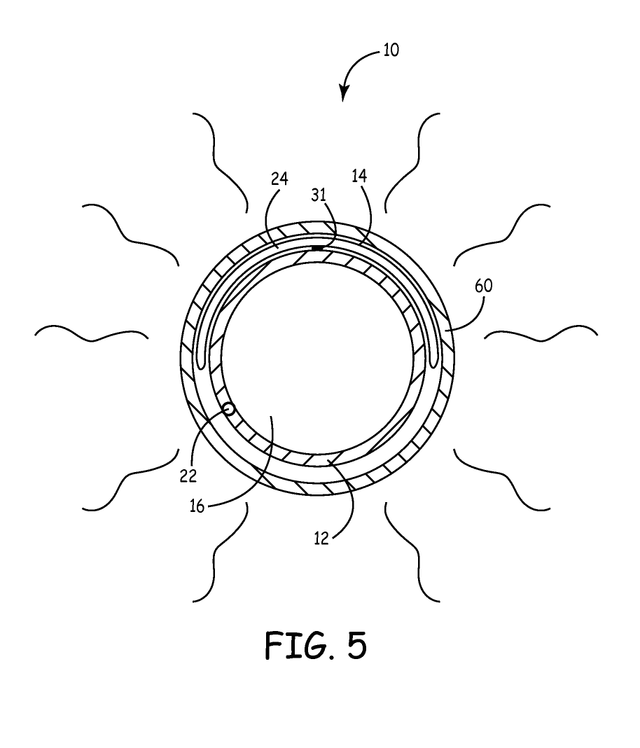

Manufacturing of the catheter system 10 will now be described with reference to FIG. 5. The first and second elongate members 12, 14 can be extruded, either separately or together. Then, the first elongate member 12 can be fixed to the second elongate member 14. As discussed above, the first and second elongate members 12, 14 can be secured using an adhesive 31.

Then, the second elongate member 14 can be collapsed so as to reduce the size of the second lumen 24. In some embodiments, the second elongate member 14 can be folded, pleated, or otherwise collapsed onto the outer surface 21 of the first elongate member 12.

Next, the first and second elongate members 12, 14 can be positioned within a removable sleeve 60 (FIG. 5) that encompasses the first and second elongate members 12, 24. In some exemplary embodiments, the removable sleeve 60 can bias and retain the second elongate member 14 in its collapsed position. Subsequently, heat can be applied to the assembly in a known heat treating process, such that the second elongate member 14 substantially retains its shape in the collapsed position. In one or more embodiments, an assembly can undergo heat treatment at 110 degrees Celsius for about 10 minutes in order to form or shape the assembly into a catheter. In one or more other embodiments, the assembly can be heat treated at about 140° F. for about 30 seconds. Also, in some embodiments, a heat source can move longitudinally relative to the sleeve 60 and elongate members 12, 24 to progressively heat localized portions of the assembly. Accordingly, the heating of the second elongate member 14 can set the second elongate member 14 in the collapsed position.

In some exemplary embodiments, the sleeve 60 remains on the first and second elongate members 12, 14 until the catheter system 10 is ready to be inserted into the patient 46 and is removed thereafter. Accordingly, the sleeve 60 can act as a packaging for the catheter system 10. In other exemplary embodiments, the sleeve 60 can be a temporary sleeve 60 that is only used during the heating step represented in the exemplary embodiment of FIG. 5. Thus, the sleeve 60 is removed after heating, and the first and second elongate members 12, 14 are placed in a different sleeve 60 for packaging purposes. In either case, the sleeve 60 is removed before the catheter system 10 is inserted into the patient 46.

Certain terminology is used herein for purposes of reference only, and thus is not intended to be limiting. For example, terms such as “upper,” “lower,” “above,” “below,” “top,” “upward,” and “downward” refer to directions in the drawings to which reference is made. Terms such as “front,” “back,” “rear,” and “side,” describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first,” “second,” and other such numerical terms referring to structures do not imply a sequence or order unless clearly indicated by the context.

When introducing elements or features and the exemplary embodiments, the articles “a,” “an,” “the” and “said” are intended to mean that there are one or more of such elements or features. The terms “comprising,” “including,” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

The description of the disclosure is merely exemplary in nature and, thus, variations that do not depart from the gist of the disclosure are intended to be within the scope of the disclosure. Such variations are not to be regarded as a departure from the spirit and scope of the disclosure.

What is claimed is:

1. A catheter system for positioning of a medical instrument, the catheter system comprising:
a first elongate member that includes a first lumen;
a second elongate member that includes a second lumen that receives the medical instrument, the second elongate member being coupled directly to the first elongate member, the second elongate member being cuttable to allow removal of the medical instrument from the second lumen, the second elongate member being selectively collapsible and expandable to change a size of the second lumen; and
a navigation tool operable for allowing detection of a location of the catheter system, the navigation tool being received in the first lumen, wherein the second elongate member is cuttable without cutting the first elongate member and the navigation tool.

2. The catheter system of claim 1, further comprising a reinforcing member that extends at least partially along the axis and reinforces the first elongate member to increase a rigidity thereof, wherein the second elongate member is cuttable without cutting a reinforcing member.

3. The catheter system of claim 2, wherein the reinforcing member is at least one of a braided member and a helical member.

4. The catheter system of claim 1, wherein the second elongate member has a wall thickness that is between about 0.0007 and 0.0015 inches.

5. The catheter system of claim 1, wherein the first elongate member includes a first longitudinal axis, and wherein the first and second elongate members are longitudinally coupled such that the second longitudinal axis extends substantially in the same direction as the first longitudinal axis.

6. The catheter system of claim 1, wherein the first elongate member includes an outer surface and the second elongate member includes an outer surface, and wherein the outer surface of the first elongate member is coupled to the outer surface of the second elongate member.

7. The catheter system of claim 1, further comprising an end member with a third lumen that is in fluid communication with the second lumen, the end member being more resistant to deformation than the second elongate member.

8. The catheter system of claim 1, further comprising a port member having a first port in fluid communication with the first lumen and a second port in fluid communication with the second lumen.

9. The catheter system of claim 8, wherein the port member further includes a third port in fluid communication with the second lumen.

10. The catheter system of claim 8, wherein the port member includes a thin walled portion that is cuttable to allow removal of the medical instrument from inside the port member.

11. The catheter system of claim 1, wherein the second elongate member is at least partially coated with a lubricant.

12. The catheter system of claim 1, further comprising a removable sleeve that encompasses the first elongate member and the second elongate member when collapsed.

13. The catheter system of claim 1, wherein the navigation tool includes an electrically conductive coil in which a current is induced in the presence of an electromagnetic field, the current being dependent on the location of the catheter system.

14. A method of manufacturing a catheter system comprising:
directly coupling an outer surface of a first elongate member having a first lumen to an outer surface of a second elongate member having a second lumen such that a longitudinal axis of the first elongate member extends substantially in the same direction as a longitudinal axis of the second elongate member;
reducing a size of the second elongate member from an expanded state to a collapsed state to reduce a size of the second lumen; and
retaining the second elongate member in the collapsed state.

15. The method of claim 14, further comprising extruding the first and second elongate members.

16. The method of claim 14, wherein directly coupling the first elongate member and the second elongate member includes coupling the first and second elongate members with an adhesive.

17. The method of claim 14, wherein retaining the second elongate member in the collapsed state includes applying heat to the second elongate member to retain the second elongate member in a substantially collapsed state.

18. The method of claim 17, wherein applying heat includes applying heat to the second elongate member at about 140° F. for about 30 seconds.

19. The method of claim 14, wherein retaining the second elongate member in the collapsed state includes encompassing the first and second elongate members in a sleeve that retains the second elongate member in the substantially collapsed state.

20. A catheter system for positioning of a medical instrument, the catheter system comprising:
a first elongate member that includes a first lumen and an outer surface;
a reinforcing member that reinforces the first elongate member;
a navigation tool with an electrically conductive coil operable for detecting a location of the catheter system, the navigation tool being received in the first lumen, the navigation tool including an electrically conductive coil in which a current is induced in the presence of an electromagnetic field, the current being dependent on the location of the catheter system;
a second elongate member that includes a second lumen that receives the medical instrument and an outer surface, the outer surface of the second elongate member being longitudinally coupled directly to the outer surface of the first elongate member, the second elongate member cuttable generally along a second longitudinal axis thereof to allow removal of the medical instrument from the second lumen, wherein the second elongate member is cuttable without cutting the first elongate member, the reinforcing member, and the navigation tool, the second elongate member being selectively collapsible and expandable to change a size of the second lumen; and
a port member having a first port in fluid communication with the first lumen and a second port in fluid communication with the second lumen, the navigation tool extending through the first port and into the first lumen, and the medical instrument extendable through the second port and into the second lumen.

21. A method of positioning a medical instrument comprising:
positioning a catheter system in a patient, the catheter system including a first elongate member that includes a first lumen and a second elongate member that includes a second lumen, the second elongate member being coupled directly to the first elongate member;
expanding the second elongate member to increase a size of the second lumen;
extending the medical instrument into the second lumen; and
segmenting the second elongate member to move the medical instrument outside the second lumen.

22. The method of claim 21, further comprising extending a navigation tool with an electrically conductive coil into the first lumen, generating an electromagnetic field relative to the coil to induce a current within the coil, and detecting the current to detect a location of the catheter system within the patient.

23. The method of claim 21, wherein expanding the second elongate member comprises introducing a fluid into the second lumen.

24. The method of claim 21, wherein expanding the second elongate member comprises pushing an inner surface of the second elongate member with the medical instrument while extending the medical instrument into the second lumen.

25. The method of claim 21, wherein segmenting the second elongate member comprises cutting the second elongate member with a blade.

26. The method of claim 21, further comprising applying heat to the second elongate member to retain the second elongate member in a substantially collapsed state.
